SEQUENCE LISTING (17 sequences)

SEQ ID NO: 1 - DNA, 620 bp, Homo sapiens
tctgaaagttgatagagagttgatagaacaatcttttcataaggtataaattctatctatttttccctaaaaacaaacaaacagcaaccattcttgcttctaattgggcagtacaatctgataggttggctagagacttgcagtggggtgtccctggtacctattcaaagactgtagctttcttctatctcattctcattttctattctttgcattgtagagttttggagcaaagaaggtcatcaaacttatacagtgagcctaacagtttccttttaagatgaggaaactgagccccagccagccatgtgattcatcacagttccttggtggctgagttgggaggagaacacacatcttctcagctcctcccactgctctttccattaagacagacagcctctcattcaaagtaagagaatttccatcatatgagcaagggacaatgagagaactgcttctcagtactcccygcttcttcctcacctacttcctcttcactggatttgtcaactcacctgtctttacgcaatagttacaatgccagcatttctctacattacatacttcagcgattctcttactggctttgcaaagtcacccaaacacgaatggaaata

SEQ ID NO: 2 - DNA, 1001 bp, Homo sapiens
agtctaaggagttctccagctgagatattctgaaagttgatagagagttgatagaacaatcttttcataaggtataaattctatctatttttccctaaaaacaaacaaacagcaaccattcttgcttctaattgggcagtacaatctgataggttggctagagacttgcagtggggtgtccctggtacctattcaaagactgtagctttcttctatctcattctcattttctattctttgcattgtagagttttggagcaaagaaggtcatcaaacttatacagtgagcctaacagtttccttttaagatgaggaaactgagccccagccagccatgtgattcatcacagttccttggtggctgagttgggaggagaacacacatcttctcagctcctcccactgctctttccattaagacagacagcctctcattcaaagtaagagaatttccatcatatgagcaagggacaatgagagaactgcttctcagtactcccygcttcttcctcacctacttcctcttcactggatttgtcaactcacctgtctttacgcaatagttacaatgccagcatttctctacattacatacttcagcgattctcttactggctttgcaaagtcacccaaacacgaatggaaatagtaaggtagagtttcagccatgagttgagttcatagctttagcaactgttaaatagctaatgtctactttctggagaataaatgctttgcaagaccctcggcaatgaaaccaaagaaagaatttaaatagcctcaccgaataattagtcagcttttcgaagtcatctcgtttctttttgttgctattgaaaaacttgacattcatgtcttccttgatggtctccacactcttttggatgctctggtcatctttaaagtttttaaaaagtttgaagtaaaaggagacaatttggctctgcattatttttctgtcactctccttggaaggaaagagcacaaacagaggatgatgt

SEQ ID NO: 3 - DNA, 28 bp, Homo sapiens
gtgaatgaagagttaatattttattagg

SEQ ID NO: 4 - DNA, 24 bp, Homo sapiens
ttcctttaaactccttaaatcctt

SEQ ID NO: 5 - DNA, 28 bp, Homo sapiens
gtgaatgaagagttaatattttattagg

SEQ ID NO: 6 - DNA, 19 bp, Homo sapiens
ggtgggtataatgggtttg

SEQ ID NO: 7 - DNA, 23 bp, Homo sapiens
ggatttaaggagtttaaaggaaa

SEQ ID NO: 8 - DNA, 24 bp, Homo sapiens
aaaacaatatactacacctcctct

SEQ ID NO: 9 - DNA, 16 bp, Homo sapiens
ttaaaaaatttgtgaa

SEQ ID NO: 10 - DNA, 30 bp, Homo sapiens
tttgggtgattttgtaaagttagtaagaga

SEQ ID NO: 11 - DNA, 24 bp, Homo sapiens
ccattaaaacaaacaacctctcat

SEQ ID NO: 12 - DNA, 20 bp, Homo sapiens
aaactacttctcaatactcc

SEQ ID NO: 13 - DNA, 20 bp, Homo sapiens
actacttctcaatagtccct

SEQ ID NO: 14 - DNA, 20 bp, Homo sapiens
actacttctcaatagtcccc

SEQ ID NO: 15 - DNA, 26 bp, Homo sapiens
tcagtactccctgtgcttcttcctca

SEQ ID NO: 16 - DNA, 26 bp, Homo sapiens
tcagtactccccgtgcttcttcctca

SEQ ID NO: 17 - DNA, 26 bp, Homo sapiens
tcagtactccccgtgcttcttcctca

(19) United States
(12) Patent Application Publication
(10) Pub. No.: US 2015/0086567 A1
(43) Pub. Date: Mar. 26, 2015

(54) ROLE OF IFNG METHYLATION IN INFLAMMATORY BOWEL DISEASE

(71) Applicant: CEDARS-SINAI MEDICAL CENTER, LOS ANGELES, CA (US)

(72) Inventors: Rebecca Gonsky, Los Angeles, CA (US); Richard Deem, Azusa, CA (US); Stephan R. Targan, Santa Monica, CA (US)

(73) Assignee: CEDARS-SINAI MEDICAL CENTER, LOS ANGELES, CA (US)

(21) Appl. No.: 14/538,673

(22) Filed: Nov. 11, 2014

Related U.S. Application Data
(63) Continuation-in-part of application No. 14/007,400, filed on Sep. 25, 2013.
(60) Provisional application No. 61/467,899, filed on Mar. 25, 2011.
(60) Provisional application No. 62/020,899, filed on Jul. 3, 2014.

(51) Int. Cl. C12Q 1/68; C07K 16/24

(52) U.S. Cl. CPC C12Q 1/6886; C07K 16/241; C12Q 2600/112
USPC 424/158.1; 435/6.11; 435/5

(57) ABSTRACT

The invention relates to method of diagnosing susceptibility to inflammatory bowel disease (IBD) in an individual by obtaining a sample from the individual, assaying the sample to determine the presence or absence of one or more risk genetic variants and/or an increase in IFNG DNA methylation. In one embodiment, the present invention provides a method of diagnosing susceptibility to inflammatory bowel disease (IBD) in an individual by obtaining a sample from the individual, assaying the sample to determine the presence or absence of one or more risk genetic variants and/or an increase in IFNG DNA methylation relative to a normal subject, and diagnosing susceptibility to inflammatory bowel disease based on the presence of one or more risk genetic variants and/or an increase in IFNG DNA methylation relative to a normal subject. In another embodiment, the IBD is ulcerative colitis.

### STATEMENT REGARDING FEDERALLY-SPONSORED RESEARCH

This invention was made with government support under Grant No. UL1RR033176 awarded by the National Center for Research Resources, Grant No. UL1TR000124 awarded by the National Center for Advancing Translational Sciences, and Grant Nos. DK043211 and DK046763 awarded by United States Public Health Service. The government has certain rights in the invention.

### FIELD OF INVENTION

The invention relates to the field of genetics and medicine. More specifically, the invention relates to methods of diagnosing and treating inflammatory bowel disease including ulcerative colitis and Crohn's disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

### SUMMARY OF THE INVENTION

Various embodiments include a method of diagnosing susceptibility to an inflammatory bowel disease (IBD) subtype in an individual, comprising obtaining a sample from the individual, assaying the sample to determine the presence or absence of at least one risk genetic variant at the genetic locus of IFNG, diagnosing susceptibility to the IBD subtype based on the presence of at least one risk genetic risk variant at the genetic locus of IFNG. In another embodiment, the IBD is ulcerative colitis. In another embodiment, the IBD is associated with early surgical intervention. In another embodiment, the IBD is associated with colitis, a small bowel disease phenotype, an aggressive complicating phenotype, an internal penetrating disease phenotype, a stricturing disease phenotype, a fibrostenosing disease phenotype, or a fistulating disease phenotype, or a combination thereof. In another embodiment, the IBD is associated with at least one risk serological marker selected from the group consisting of ANCA, ASCA, anti-Cbir1, anti-I2, and anti-OmpC. In another embodiment, the at least one risk genetic variant is a “T” allele of SEQ. ID. NO.: 1. In another embodiment, the at least one risk genetic variant is associated with a lower level of IFNG DNA methylation relative to a healthy subject. In another embodiment, the at least one risk genetic variant is associated with a higher level of anti-Cbir1 relative to a healthy subject. In another embodiment, the at least one risk genetic variant is a “C” allele of SEQ. ID. NO.: 1. In another embodiment, the at least one risk genetic variant is associated with a higher level of IFNG DNA methylation relative to a healthy subject.

Other embodiments include a method of diagnosing inflammatory bowel disease (IBD) in an individual, comprising obtaining a sample from an individual, assaying the sample to determine the presence or absence of at least one risk genetic variant at the genetic locus of IFNG, assaying the sample to determine an increase or decrease in IFNG DNA methylation relative to a healthy subject, and diagnosing IBD in the individual based on the presence of at least one risk genetic variant at the genetic locus of IFNG and an increase in IFNG DNA methylation relative to a healthy subject. In another embodiment, the IBD is Crohn's disease or ulcerative colitis. In another embodiment, the at least one risk genetic variant is a “T” allele of SEQ. ID. NO.: 1. In another embodiment, the method further comprises determining the presence of a high level of anti-Cbir1 relative to a healthy subject. In another embodiment, the IBD is associated with severe ulcerative colitis conditions. In another embodiment, the IBD is associated with colitis, a small bowel disease phenotype, an aggressive complicating phenotype, an internal penetrating disease phenotype, a stricturing disease phenotype, a fibrostenosing disease phenotype, or a fistulating disease phenotype, or a combination thereof. In another embodiment, the IBD is associated with at least one risk serological marker selected from the group consisting of ANCA, ASCA, anti-Cbir1, anti-I2, and anti-OmpC. In another embodiment, the sample comprises a nucleic acid from the individual. In another embodiment, the sample is a body fluid. In another embodiment, the body fluid is whole blood, plasma, saliva, mucus, or cheek swab. In another embodiment, the sample is a cell or tissue. In another embodiment, the cell , wherein the cell is a lymphoblastoid cell line obtained from the individual and transformed with an Epstein Barr virus. In another embodiment, the cell is a mucosal T cell, a lamina propria T cell, or a peripheral blood T cell.

Other embodiments include a method of treating an inflammatory bowel disease (IBD) in an individual, comprising obtaining a sample from an individual, assaying the sample to determine the presence of at least one risk genetic variant at the genetic locus of IFNG, assaying the sample to determine an aberrant level of IFNG DNA methylation, and treating the IBD in the individual. In another embodiment, the IBD is Crohn's disease or ulcerative colitis. In another embodiment, the IBD is associated with early surgical intervention. In another embodiment, the IBD is associated with colitis, a small bowel disease phenotype, an aggressive complicating phenotype, an internal penetrating disease phenotype, a stricturing disease phenotype, a fibrostenosing disease phenotype, or a fistulating disease phenotype, or a combination thereof. In another embodiment, the at least one risk genetic variant at the genetic locus of IFNG is SEQ. ID. NO.: 1.

Various embodiments include a method of treating an inflammatory bowel disease (IBD) in an individual, comprising, obtaining a sample from the individual, assaying the sample to detect the presence or absence of at least one risk genetic variant at the genetic locus of IFNG, and/or assaying the sample to detect an increase or decrease in IFNG DNA methylation relative to a healthy individual, and treating the IBD in the individual. In another embodiment, the IBD comprises Crohn's disease (CD) or ulcerative colitis (UC). In another embodiment, the IBD is associated with colitis, a small bowel disease phenotype, a complicated disease phenotype, an internal penetrating disease phenotype, a penetrating disease phenotype, a stricturing disease phenotype, a fibrostenosing disease phenotype, a fistulating disease phenotype, a severe disease course, or an aggressive disease course, or a combination thereof. In another embodiment, the individual is a human. In another embodiment, the sample comprises a nucleic acid from the individual. In another embodiment, the sample comprises a body fluid, cheek swab, mucus, whole blood, blood, serum, plasma, urine, saliva, semen, lymph, fecal extract, or sputum, or a combination thereof. In another embodiment, the sample comprises a cell or tissue. In another embodiment, the cell is a lymphoblastoid cell line obtained from the individual and transformed with an Epstein Barr virus. In another embodiment, the cell is a mucosal T cell, a lamina propria T cell, or a peripheral blood T cell. In another embodiment, the at least one risk genetic variant is the “T” allele of SEQ ID NO:1 or SEQ ID NO:2. In another embodiment, IFNG DNA methylation is IFNG promoter methylation. In another embodiment, further comprising assaying the sample to detect an increase of INF-γ secretion relative to a healthy individual. In another embodiment, further comprising assaying the sample to detect an increase or decrease of at least one risk serological marker relative to a healthy individual, wherein the at least one risk serological marker is selected from the group consisting of ANCA, ASCA, anti-Cbir1, anti-I2, and anti-OmpC. In another embodiment, treating the IBD comprises conducting colectomy on the individual, upon detecting the presence of at least one risk genetic variant at the genetic locus of IFNG and/or a decrease in IFNG DNA methylation relative to a healthy individual. In another embodiment, treating the IBD comprises administering a TNF signaling inhibitor to the individual, upon detecting the absence of at least one risk genetic variant at the genetic locus of IFNG and/or an increase in IFNG DNA methylation relative to a healthy individual. In another embodiment, the TNF signaling inhibitor comprises an anti-TNF antibody. In another embodiment, the TNF signaling inhibitor comprises infliximab, adalimumab, certolizumab, certolizumab pegol, golimumab, etanercept, or onercept, or a combination thereof. In another embodiment, the TNF signaling inhibitor is administered topically, intravascularly, intravenously, intraarterially, intramuscularly, subcutaneously, intraperitoneally, intranasally, or orally. IN another embodiment, the TNF signaling inhibitor is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In another embodiment, the TNF signaling inhibitor is administered about 1-3 times per day, 1-7 times per week, or 1-9 times per month. In another embodiment, the TNF signaling inhibitor is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

Other embodiments include a method of treating an inflammatory bowel disease (IBD) in an individual, comprising genotyping the individual for a risk genetic variant at the genetic locus of IFNG, and if the individual is positive for the risk genetic variant, conducting colectomy on the individual, and if the individual is negative for the risk genetic variant, administering a TNF signaling inhibitor to the individual. Other embodiments include a method of treating an inflammatory bowel disease (IBD) in an individual, comprising obtaining a sample from the individual, contacting the sample with an oligonucleotide probe specific to a risk genetic variant at the genetic locus of IFNG, forming an allele-specific hybridization complex between the oligonucleotide probe and the risk genetic variant, detecting the allele-specific hybridization complex, and if the allele-specific hybridization complex is detected, conducting colectomy on the individual, and if the allele-specific hybridization complex is not detected, administering a TNF signaling inhibitor to the individual. In another embodiment, the individual is a human. In another embodiment, the risk genetic variant is the “T” allele of SEQ ID NO:1 or SEQ ID NO:2. In another embodiment, the oligonucleotide probe is labeled with a fluorescent dye, and wherein detecting the allele-specific hybridization complex comprises detecting fluorescence signal from the oligonucleotide probe. In another embodiment, the oligonucleotide probe comprises a reporter dye and a quencher dye. In another embodiment, further comprising conducting PCR amplification after forming the allele-specific hybridization complex. In another embodiment, detecting the allele-specific hybridization complex comprises detecting the electrophoretic mobility of the allele-specific hybridization complex.

Other features and advantages of the invention will become apparent from the following detailed description, taken in conjunction with the accompanying drawings, which illustrate, by way of example, various embodiments of the invention.

### BRIEF DESCRIPTION OF THE FIGURES

FIG. 1 depicts, in accordance with various embodiments of the invention, allele specific differential methylation associated with the +2167 but not +2209 CpG site.

FIG. 2 depicts, in accordance with various embodiments of the invention, IFNG SNP is functionally associated with enhanced promoter methylation and decreased protein expression.

FIG. 3 depicts, in accordance with various embodiments of the invention, IFNG SNP is associated with increased time to surgery and decreased Cbir responsiveness.

FIG. 4 depicts, in accordance with various embodiments of the invention, enhanced nucleoprotein binding to IFNG rs1861494 “T” allele compared to “C” allele.

FIG. 5 depicts, in accordance with various embodiments of the invention, enhanced nucleoprotein binding methylated CpG.

FIG. 6 depicts, in accordance with various embodiments of the invention, a chart summarizing the IFNG research findings.

FIG. 7 depicts, in accordance with various embodiments of the present invention, schematic of IFNG gene showing conserved regions between human and mouse.

FIG. 8 depicts, in accordance with various embodiments of the present invention, relationship of rs1861494 to serological markers and time to surgery. (A), rs1861494 SNP allele distribution in IBD patients as a function of sero-reactivity to ANCA; (B), rs1861494 T allele association with ANCA EU in UC patients (n=17 for C, n=39 for T); (C), Time to surgery curve of medically refractive UC patients based on rs1861494 allele genotype (n=13 for C, n=153 for T). Bars represent means±SEM.

FIG. 9 depicts, in accordance with various embodiments of the present invention, rs1861494 T allele, compared to C allele, genotype is associated with more severe disease behavior in CD patients. Frequency of non-stricturing/penetrating vs. complicated (stricturing/penetrating) disease phenotype was determined in 44 CD patients.

FIG. 10 depicts, in accordance with various embodiments of the present invention, secretion of IFN gamma from IBD T cells stimulated with anti-CD3 antibody for 24 h (n=28 for C, n=104 for T). Bars represent means ±SEM.

FIG. 11 depicts, in accordance with various embodiments of the present invention, allele/strand specific methylation of IBD patients heterozygous for rs1861494. (A), rs1861494; (B), CpG at +2052 and +2007; (C), Correlation of methylation of rs1861494 with methylation index of IFNG promoter (CpG sites at −54, −186, and −295 bp) (n=36). Bars represent means±SEM.

FIG. 12 depicts, in accordance with various embodiments of the present invention, EMSA analysis of rs1861494 regulatory binding proteins. PBMC were stimulated up to 4 h with PMA/ionomycin and nuclear protein extracts were obtained. EMSAs were performed for binding activity to the (A), C or T SNP; (B) kinetics of nucleo-protein binding to the non-methlyated (C SNP), asymmetrically methylated (one strand, C/MC SNP) or symmetrically methylated (both strands, MC-MC SNP) oligonucleotide. Representative of 4 experiments.

FIG. 13 depicts, in accordance with various embodiments of the invention, various genetic loci as part of a correlation analysis of IFNG expression.

FIG. 14 depicts, in accordance with various embodiments herein, genes found to be associated with both DMR and eQTL.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“IBD” as used herein is an abbreviation of inflammatory bowel disease.

“CD” as used herein is an abbreviation of Crohn's Disease.

“SNP” as used herein is an abbreviation of single nucleotide polymorphism.

As used herein, the term “IFNG” refers to the gene encoding IFN-gamma. Similarly, “IFNG production,” or “IFNG secretion” refers to the product expressed from the IFNG genetic locus.

An example of SNP rs1861494 is provided herein as SEQ. ID. NO.: 1 and SEQ. ID. NO.: 2.

“TNF” as used herein is an abbreviation of tumor necrosis factor.

As used herein, the term “TNF signaling inhibitor” (also interchangeably called as TNF blocker or inhibitor, anti-TNF reagent, agent, drug or therapeutic,) refers to any reagent that suppress responses to TNF and/or inhibits the TNF signaling, including inhibition of any molecular signaling step from the TNF ligand through its receptor to various downstream target molecules. A TNF signaling inhibitor can be a small molecule; a nucleic acid such as siRNA, shRNA, and miRNA; a nucleic acid analogue such as PNA, pc-PNA, and LNA; an aptamer; a ribosome; a peptide; a protein; an avimer; an antibody, or variants and fragments thereof. Examples of the TNF signaling inhibitor include but are not limited to CDP571, CDP860, CDP870, infliximab, adalimumab, certolizumab, certolizumab pegol, golimumab, etanercept, onercept, MAP kinase inhibitors, xanthine derivatives (e.g. pentoxifylline), bupropion, 5-HT2A agonist hallucinogens including (R)-DOI, TCB-2, LSD and LA-SS-Az, curcumin, and catechins

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As disclosed herein, the inventors determined what was the methylation status for IFNG rs1861494 SNP alleles and whether a functional relationship exists between allele specific methylation and gene expression. 154 IBD patients were genotyped for the IFNG rs1861494. DNA strand specific methylation levels for SNP +2109 and adjacent +2167 and +2209 CpG sites were determined by pyrosequencing. Allele and methylation-specific nucleo-protein binding was determined by EMSA. Levels of IFNG secretion and immune response to CBir were measured by ELISA.

As further disclosed herein, the wt rs1861494 T allele is un-methylated whereas the C allele displays 55% methylation. In adjacent CpG sites allele-specific DNA methylation was noted at the +2167, but not +2209, with decreased methylation of the C vs. T SNP allele DNA strands (p<0.001). The rs1861494 IFNG polymorphism is functionally associated with decreased IFNG production and levels of immune response to CBir. Allele-specific and methylation-sensitive alteration in DNA trans-factor binding patterns to the SNP was noted. Nucleo-protein binding to the unmethylated C SNP was lower than that seen for T SNP. However, methylation of the C allele strand markedly enhanced binding and the appearance of an additional nucleo-protein complex. These results link the same cis-regulatory IFNG variant with modulation of DNA strand methylation and transcription factor binding supporting a functional role for rs1861494 gene variant in regulating IFNG expression.

In one embodiment, the present invention provides a method of diagnosing susceptibility to inflammatory bowel disease (IBD) in an individual by obtaining a sample from the individual, assaying the sample to determine the presence or absence of one or more risk genetic variants and/or an increase in IFNG DNA methylation relative to a normal subject, and diagnosing susceptiblity to inflammatory bowel disease based on the presence of one or more risk genetic variants and/or an increase in IFNG DNA methylation relative to a normal subject. In another embodiment, the IBD is ulcerative colitis. In another embodiment, the one ore more risk genetic variants include SNP rs1861494 with a “C” allele. In another embodiment, the presence of one or more risk genetic variants and/or increase in IFNG DNA methylation relative to a normal subject is associated with a decrease in levels of IFNG expressed relative to levels found in a healthy person.

In one embodiment, the present invention provides a method of diagnosing susceptibility to inflammatory bowel disease (IBD) in an individual by obtaining a sample from the individual, assaying the sample to determine the presence or absence of one or more risk genetic variants and/or a decrease in IFNG DNA methylation relative to a normal subject, and diagnosing susceptiblity to inflammatory bowel disease based on the presence of one or more risk genetic variants and/or an decrease in IFNG DNA methylation relative to a normal subject. In another embodiment, the IBD is ulcerative colitis. In another embodiment, the one or more risk genetic variants include SNP rs1861494 with a “T” allele. In another embodiment, the presence of one or more risk genetic variants and/or decrease in IFNG DNA methylation relative to a normal subject is associated with a increase in levels of IFNG protein relative to levels found in a healthy person.

In one embodiment, the present invention provides a method of treating IBD in an individual by determining the presence of aberrant DNA methylation patters at the IFNG genetic locus, relative to a healthy subject, and treating the individual.

As described herein, the inventors explored the association of rs1861494 T/C SNP with severity of disease in IBD and found a significant association of the T allele to severity in both UC and CD. Furthermore, the rs1861494 T allele functionally correlated with increased IFN-gamma expression. In this context, the rs1861494 T/C polymorphism introduces a new CpG dinucleotide sequence that serves as an epigenetic target for DNA methylation resulting in altered transcription factor binding to this region that might have a functional consequence on transcription of IFN-gamma expression.

Mucosal expression of IFN-γ plays a pivotal role in IBD pathogenesis and IBD-risk regions flank IFNG. The conserved IFNG rs1861494 T/C, introduces a new CpG methylation site, and is associated with disease severity and lack of therapeutic response in other infectious and immune mediated disorders, and is in linkage-disequilibrium with a UC disease severity region. It seems likely that CpG-altering SNPs modify methylation and gene expression. This study evaluated the association between rs1861494 and clinical, serologic and methylation patterns in IBD patients.

Peripheral T cells of UC and CD patients were genotyped for rs1861494 and analyzed for allele-specific and IFNG promoter methylation. Serum ANCA and IFN-γ secretion were measured by ELISA and nucleo-protein complex formation by EMSA.

IFNG rs1861494 T allele carriage in IBD patients was associated with enhanced secretion of IFN-γ. T allele carriage was associated in UC with high levels of ANCA and faster progression to colectomy. In CD, it was associated with complicated disease involving a stricturing/penetrating phenotype. Likewise, IFNG rs1861494 displayed genotype specific modulation of DNA methylation and transcription factor complex formation.

This study reports the first association of IFNG rs1861494 T allele with enhanced IFN-γ secretion and known IBD clinical parameters indicative of more aggressive disease, as well as serological markers associated with treatment resistance to anti-TNF therapy in IBD patients. These data is useful prognostically as predictors of early response to anti-TINF therapy to identify IBD patients for improved personalized therapeutics.

A variety of methods can be used to detect the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well-known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well-known assays described above or another art-recognized genetic assay.

### Treatment Methods and Systems

In various embodiments, the present invention provides a method of administering a TNF signaling inhibitor to an individual. In one embodiment, the individual has already been diagnosed with an inflammatory bowel disease (IBD). In some embodiments, the method may consist of or may consist essentially of or may comprise: (a) genotyping the individual for a risk genetic variant at the genetic locus of IFNG; and (b) if the individual is negative for the risk genetic variant, administering the TNF signaling inhibitor to the individual, and if the individual is positive for the risk genetic variant, not administering the TNF signaling inhibitor to the individual. In other embodiments, the method may consist of or may consist essentially of or may comprise: (a) obtaining a sample from the individual; (b) contacting the sample with an oligonucleotide probe specific to a risk genetic variant at the genetic locus of IFNG; (c) forming an allele-specific hybridization complex between the oligonucleotide probe and the risk genetic variant; (d) detecting the allele-specific hybridization complex; and (e) if the allele-specific hybridization complex is not detected, administering the TNF signaling inhibitor to the individual, and if the allele-specific hybridization complex is detected, not administering the TNF signaling inhibitor to the individual. In various embodiments, the method further comprises conducting PCR amplification after forming the allele-specific hybridization complex.

In various embodiments, the present invention provides a method of treating, preventing, reducing the likelihood of having, reducing the severity of and/or slowing the progression of an inflammatory bowel disease (IBD) in an individual. In some embodiments, the method may consist of or may consist essentially of or may comprise: (a) genotyping the individual for a risk genetic variant at the genetic locus of IFNG; and (b) if the individual is positive for the risk genetic variant, conducting colectomy on the individual, and if the individual is negative for the risk genetic variant, administering a TNF signaling inhibitor to the individual. In other embodiments, the method may consist of or may consist essentially of or may comprise: (a) obtaining a sample from the individual; (b) contacting the sample with an oligonucleotide probe specific to a risk genetic variant at the genetic locus of IFNG; (c) forming an allele-specific hybridization complex between the oligonucleotide probe and the risk genetic variant; (d) detecting the allele-specific hybridization complex; and (e) if the allele-specific hybridization complex is detected, conducting colectomy on the individual, and if the allele-specific hybridization complex is not detected, administering a TNF signaling inhibitor to the individual. In various embodiments, the method further comprises conducting PCR amplification after forming the allele-specific hybridization complex.

In various embodiments, detecting the allele-specific hybridization complex comprises detecting the electrophoretic mobility of the allele-specific hybridization complex. In another embodiment, the oligonucleotide probe is labeled with a fluorescent dye. In some embodiments, the oligonucleotide probe comprises a reporter dye and a quencher dye. In still another embodiment, detecting the allele-specific hybridization complex comprises detecting fluorescence signal from the oligonucleotide probe.

In various embodiments, the present invention also provides a system for administering a TNF signaling inhibitor to an individual and/or for treating, preventing, reducing the likelihood of having, reducing the severity of and/or slowing the progression of an inflammatory bowel disease (IBD) in an individual. The system may consist of or may consist essentially of or may comprise: (a) an oligonucleotide probe specific to a risk genetic variant at the genetic locus of IFNG; and (b) a TNF signaling inhibitor. In one embodiment, the system further comprises a module configured for conducting PCR amplification. In another embodiment, the oligonucleotide probe is labeled with a fluorescent dye. In some embodiments, the oligonucleotide probe comprises a reporter dye and a quencher dye. In still another embodiment, the system further comprises a module configured to detecting fluorescence signal from the oligonucleotide probe.

In various embodiments, the present invention provides a method of treating, preventing, reducing the likelihood of having, reducing the severity of and/or slowing the progression of an inflammatory bowel disease (IBD) in an individual. The method may consist of or may consist essentially of or may comprise: (a) obtaining a sample from the individual; (b) assaying the sample to detect the presence or absence of at least one risk genetic variant at the genetic locus of IFNG, and/or assaying the sample to detect an increase or decrease in IFNG DNA methylation relative to a healthy individual; and (c) treating the IBD in the individual. In some embodiments, the method may consist of or may consist essentially of or may comprise: (a) obtaining a sample from the individual; (b) assaying the sample to detect the presence or absence of at least one risk genetic variant at the genetic locus of IFNG; and (c) treating the IBD in the individual. In other embodiments, the method may consist of or may consist essentially of or may comprise: (a) obtaining a sample from the individual; (b) assaying the sample to detect an increase or decrease in IFNG DNA methylation relative to a healthy individual; and (c) treating the IBD in the individual. In various embodiments, the method may consist of or may consist essentially of or may comprise: (a) obtaining a sample from the individual; (b) assaying the sample to detect the presence or absence of at least one risk genetic variant at the genetic locus of IFNG, and assaying the sample to detect an increase or decrease in IFNG DNA methylation relative to a healthy individual; and (c) treating the IBD in the individual. In some embodiments, treating the IBD comprises conducting colectomy on the individual, upon detecting the presence of at least one risk genetic variant at the genetic locus of IFNG and/or a decrease in IFNG DNA methylation relative to a healthy individual. In other embodiments, treating the IBD comprises administering a TNF signaling inhibitor to the individual, upon detecting the absence of at least one risk genetic variant at the genetic locus of IFNG and/or an increase in IFNG DNA methylation relative to a healthy individual.

In various embodiments, the IBD comprises Crohn's disease (CD) or ulcerative colitis (UC). In some embodiments, the IBD is associated with colitis, a small bowel disease phenotype, a complicated disease phenotype, an internal penetrating disease phenotype, a penetrating disease phenotype, a stricturing disease phenotype, a fibrostenosing disease phenotype, a fistulating disease phenotype, a severe disease course, or an aggressive disease course, or a combination thereof.

In various embodiments, the individual is a human. In some embodiments, the individual is a mammalian subject including but not limited to human, monkey, ape, dog, cat, cow, horse, goat, pig, rabbit, mouse and rat.

In various embodiments, the sample comprises a nucleic acid from the individual. In some embodiments, the sample comprises a body fluid, cheek swab, mucus, whole blood, blood, serum, plasma, urine, saliva, semen, lymph, fecal extract, or sputum, or a combination thereof. In other embodiments, the sample comprises a cell or tissue. In accordance with the present invention, the cell can be a lymphoblastoid cell line obtained from the individual and transformed with an Epstein Barr virus. Still in accordance with the present invention, the cell can be a mucosal T cell, a lamina propria T cell, or a peripheral blood T cell.

In various embodiments, the risk genetic variant is the “T” allele SNP rs1861494 (for non-limiting examples, SEQ ID NO:1 and SEQ ID NO:2 herein). In various embodiments, IFNG DNA methylation is IFNG promoter methylation.

In some embodiments, the treatment methods described herein further comprise assaying the sample to detect an increase of INF-γ secretion relative to a healthy individual. In further embodiments, treating the IBD comprises conducting colectomy on the individual, upon detecting an increase of INF-γ secretion relative to a healthy individual, or administering a TNF signaling inhibitor to the individual, upon detecting no increase of INF-γ secretion relative to a healthy individual.

In other embodiments, the treatment methods described herein further comprise assaying the sample to detect an increase or decrease of at least one risk serological marker relative to a healthy individual, wherein the at least one risk serological marker is selected from the group consisting of ANCA, ASCA, anti-Cbir1, anti-I2, and anti-OmpC. In further embodiments, treating the IBD comprises conducting colectomy on the individual, upon detecting an increase of the at least one risk serological marker relative to a healthy individual, or administering a TNF signaling inhibitor to the individual, upon detecting no increase of the at least one risk serological marker relative to a healthy individual. In one embodiment, the at least one risk serological marker is ANCA.

In some embodiments, the TNF signaling inhibitor comprises an anti-TNF antibody. In various embodiments, the TNF signaling inhibitor comprises infliximab, adalimumab, certolizumab, certolizumab pegol, golimumab, etanercept, or onercept, or a combination thereof.

Typical dosages of an effective amount of the TNF signaling inhibitor can be in the ranges recommended by the manufacturer where known therapeutic molecules or compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the TNF signaling inhibitor may be administered once a day (SID/QD), twice a day (BID), three times a day (TID), four times a day (QID), or more, so as to administer an effective amount of the TNF signaling inhibitor to the individual, where the effective amount is any one or more of the doses described herein.

In various embodiments, the TNF signaling inhibitor is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In various embodiments, the TNF signaling inhibitor is administered once, twice, three or more times. In some embodiments, the TNF signaling inhibitor is administered about 1-3 times per day, 1-7 times per week, or 1-9 times per month. Still in some embodiments, the TNF signaling inhibitor is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. Here, “mg/kg” refers to mg per kg body weight of the individual. In certain embodiments, the TNF signaling inhibitor is administered to a human.

In accordance with the invention, the TNF signaling inhibitor may be administered using the appropriate modes of administration, for instance, the modes of administration recommended by the manufacturer. In accordance with the invention, various routes may be utilized to administer the TNF signaling inhibitor of the claimed methods, including but not limited to aerosol, nasal, oral, transmucosal, transdermal, parenteral, enteral, topical, local, implantable pump, continuous infusion, capsules and/or injections. In various embodiments, the TNF signaling inhibitor is administered topically, intravascularly, intravenously, intraarterially, intramuscularly, subcutaneously, intraperitoneally, intranasally, or orally.

### Methods and Systems for Diagnosis or Prognosis

In various embodiments, the present invention provides a method of diagnosing an inflammatory bowel disease (IBD) subtype in an individual. The method may consist of or may consist essentially of or may comprise: (a) obtaining a sample from an individual; (b) assaying the sample to detect the presence or absence of at least one risk genetic variant at the genetic locus of IFNG, and/or assaying the sample to detect an increase or decrease in IFNG DNA methylation relative to a healthy individual; and (c) diagnosing the IBD subtype in the individual based on the presence of at least one risk genetic variant at the genetic locus of IFNG and/or an decrease in IFNG DNA methylation relative to a healthy individual.

In various embodiments, the present invention provides a method of predicting susceptibility to an inflammatory bowel disease (IBD) subtype in an individual. The method may consist of or may consist essentially of or may comprise: (a) obtaining a sample from the individual; (b) assaying the sample to detect the presence or absence of at least one risk genetic variant at the genetic locus of IFNG, and/or assaying the sample to detect an increase or decrease in IFNG DNA methylation relative to a healthy individual; and (c) predicting susceptibility to the IBD subtype in the individual based on the presence of at least one risk genetic variant at the genetic locus of IFNG and/or an decrease in IFNG DNA methylation relative to a healthy individual.

In various embodiments, the present invention also provides a system for diagnosing or predicting susceptibility to an inflammatory bowel disease (IBD) subtype in an individual. The system may consist of or may consist essentially of or may comprise an oligonucleotide probe specific to a risk genetic variant at the genetic locus of IFNG. In various embodiments, the risk genetic variant is the “T” allele SNP rs1861494. In one embodiment, the system further comprises a module configured for conducting PCR amplification. In another embodiment, the oligonucleotide probe is labeled with a fluorescent dye. In some embodiments, the oligonucleotide probe comprises a reporter dye and a quencher dye. In still another embodiment, the system further comprises a module configured to detecting fluorescence signal from the oligonucleotide probe.

In various embodiments, the IBD subtype comprises Crohn's disease (CD), ulcerative colitis (UC), or medically refractory UC (MR-UC). In one embodiment, the IBD subtype is associated with early surgical intervention or faster progression to colectomy. In another embodiment, the IBD subtype is associated with poor response, no response, and/or resistance to anti-TNF therapy. In some embodiments, the IBD subtype is associated with colitis, a small bowel disease phenotype, a complicated disease phenotype, an internal penetrating disease phenotype, a penetrating disease phenotype, a stricturing disease phenotype, a fibrostenosing disease phenotype, a fistulating disease phenotype, a severe disease course, or an aggressive disease course, or a combination thereof.

In various embodiments, the IBD subtype is associated with at least one risk serological marker selected from the group consisting of ANCA, ASCA, anti-Cbir1, anti-I2, and anti-OmpC.

In various embodiments, the individual is a human. In some embodiments, the individual is a mammalian subject including but not limited to human, monkey, ape, dog, cat, cow, horse, goat, pig, rabbit, mouse and rat.

In various embodiments, the sample comprises a nucleic acid from the individual. In some embodiments, the sample comprises a body fluid, cheek swab, mucus, whole blood, blood, serum, plasma, urine, saliva, semen, lymph, fecal extract, or sputum, or a combination thereof. In other embodiments, the sample comprises a cell or tissue. In accordance with the present invention, the cell can be a lymphoblastoid cell line obtained from the individual and transformed with an Epstein Barr virus. Still in accordance with the present invention, the cell can be a mucosal T cell, a lamina propria T cell, or a peripheral blood T cell.

In various embodiments, the risk genetic variant is the “T” allele SNP rs1861494. In various embodiments, IFNG DNA methylation is IFNG promoter methylation. In one embodiment, the at least one risk genetic variant is associated with a decrease of IFNG DNA methylation relative to a healthy individual.

In one embodiment, the at least one risk genetic variant is associated with an increase of INF-γ secretion relative to a healthy individual. In another embodiment, the methods described herein further comprise assaying the sample to detect an increase of INF-γ secretion relative to a healthy individual. In still another embodiment, the methods described herein further comprise predicting susceptibility to and/or diagnosing the IBD subtype in the individual based on an increase of INF-γ secretion relative to a healthy individual.

In one embodiment, the at least one risk genetic variant is associated with an increase of ANCA relative to a healthy individual. In another embodiment, the methods described herein further comprise assaying the sample to detect an increase of ANCA relative to a healthy individual. In still another embodiment, the methods described herein further comprise predicting susceptibility to and/or diagnosing the IBD subtype in the individual based on an increase of ANCA relative to a healthy individual.

In one embodiment, the at least one risk genetic variant is associated with an increase of at least one risk serological marker relative to a healthy individual, wherein the at least one risk serological marker is selected from the group consisting of ANCA, ASCA, anti-Cbir1, anti-I2, and anti-OmpC. In another embodiment, the methods described herein further comprise assaying the sample to detect an increase or decrease of at least one risk serological marker relative to a healthy individual, wherein the at least one risk serological marker is selected from the group consisting of ANCA, ASCA, anti-Cbir1, anti-I2, and anti-OmpC. In still another embodiment, the methods described herein further comprise predicting susceptibility to and/or diagnosing the IBD subtype in the individual based on an increase of at least one risk serological marker relative to a healthy individual.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Methylation

Epigenetic remodeling of chromatin via DNA methylation affects transcriptional activation. It has been demonstrated a distinct IFNG DNA methylation pattern in mucosal T cells from IBD patients and in peripheral T cells of a subset of UC patients. Decreased IFNG methylation was associated with increased IFNG production and seroreactivity to microbial antigens. GWA Studies identified UC-risk/severity regions linked to single nucleotide polymorphisms (SNP) flanking IFNG. One of the challenges of GWAS is to define the functional consequences of these genetic variations. Many disease-associated SNPs target CpG sites, which are relatively rare within the genome and serve as sites for DNA methylation. Recently, allele specific methylation was reported to preferentially occur at CpG sites adjacent to SNPs that alter CpG sites. The CpG (C/T) SNP rs1861494 (+2109) is located in a conserved regulatory region of the third intron of IFNG, within the same LD block implicated with UC and disease severity. Two adjacent CpG sites are found at +2167 and +2209 bp. Though typically both alleles contribute towards gene expression, monoallelic expression of IFNG protein has been reported. Moreover, it seems likely that variants that alter CpG sites not only alter methylation but may lead to unequal allelic expression.

The inventors determined what was the methylation status for IFNG rs1861494 SNP alleles and whether a functional relationship exists between allele specific methylation and gene expression. 154 IBD patients were genotyped for the IFNG rs1861494. DNA strand specific methylation levels for SNP +2109 and adjacent +2167 and +2209 CpG sites were determined by pyrosequencing. Allele and methylation-specific nucleo-protein binding was determined by EMSA. Levels of IFNG secretion and immune response to CBir were measured by ELISA.

The wt rs1861494 T allele is un-methylated whereas the C allele displays 55% methylation. In adjacent CpG sites allele-specific DNA methylation was noted at the +2167, but not +2209, with decreased methylation of the C vs. T SNP allele DNA strands (p<0.001). The rs1861494 IFNG polymorphism is functionally associated with decreased IFNG production and levels of immune response to CBir. Allele-specific and methylation-sensitive alteration in DNA trans-factor binding patterns to the SNP was noted. Nucleo-protein binding to the unmethylated C SNP was lower than that seen for T SNP. However, methylation of the C allele strand markedly enhanced binding and the appearance of an additional nucleo-protein complex. These results link the same cis-regulatory IFNG variant with modulation of DNA strand methylation and transcription factor binding supporting a functional role for rs1861494 gene variant in regulating IFNG expression.

### Example 2

### Methylation Clusters and CD Subgroups

The inventors identified distinct genome-wide methylation and RNA expression patterns in IBD. Matched CD3+ LPL and PBL were isolated from 12 CD, 11 UC or 8 normal (NL) donors; DNA and RNA was extracted; differentially methylated regions (DMRs) were identified using Illumina 450 k Infinium Bead Array; mRNA expression measured using Illumina expression array; data analysis using BRB Array Tools; Data was filtered 80% variance, less than 50% missing, and 2-fold differences across all samples, yielding 13,079 CpG sites. As a result, the inventors found genome-wide differentially methylated regions in mucosal vs. peripheral T cells, and in IBD compared to normal patients. The CD patients display a greater percentage of DMRs mapped within IBD GWAS vs. non-GWAS loci, and distinct methylation and mRNA expression profiles in patients requiring early surgical intervention. Combined epigenetic and expression profiling may stratify CD patients into distinct subgroups exhibiting molecular patterns that identify patients with different natural history of disease.

### Example 3

Epigenetic remodeling of chromatin via DNA methylation affects transcriptional activation. It has been demonstrated a distinct IFNG DNA methylation pattern in mucosal T cells from IBD patients and in peripheral T cells of a subset of UC patients. Decreased IFNG methylation was associated with increased IFNG production and seroreactivity to microbial antigens. GWA Studies identified UC-risk/severity regions linked to single nucleotide polymorphisms (SNP) flanking IFNG. One of the challenges of GWAS is to define the functional consequences of these genetic variations. Many disease-associated SNPs target CpG sites, which are relatively rare within the genome and serve as sites for DNA methylation. Recently, allele specific methylation was reported to preferentially occur at CpG sites adjacent to SNPs that alter CpG sites. The CpG (C/T) SNP rs1861494 (+2109) is located in a conserved regulatory region of the third intron of IFNG, within the same LD block implicated with UC and disease severity. Two adjacent CpG sites are found at +2167 and +2209 bp. Though typically both alleles contribute towards gene expression, monoallelic expression of IFNG protein has been reported. Moreover, it seems likely that variants that alter CpG sites not only alter methylation but may lead to unequal allelic expression.

The inventors determined what was the methylation status for IFNG rs1861494 SNP alleles and whether a functional relationship exists between allele specific methylation and gene expression. 154 IBD patients were genotyped for the IFNG rs1861494. DNA strand specific methylation levels for SNP +2109 and adjacent +2167 and +2209 CpG sites were determined by pyrosequencing. Allele and methylation-specific nucleo-protein binding was determined by EMSA. Levels of IFNG secretion and immune response to CBir were measured by ELISA.

The wt rs1861494 T allele is un-methylated whereas the C allele displays 55% methylation. In adjacent CpG sites allele-specific DNA methylation was noted at the +2167, but not +2209, with decreased methylation of the C vs. T SNP allele DNA strands (p<0.001). The rs1861494 IFNG polymorphism is functionally associated with decreased IFNG production and levels of immune response to CBir. Allele-specific and methylation-sensitive alteration in DNA trans-factor binding patterns to the SNP was noted. Nucleo-protein binding to the unmethylated C SNP was lower than that seen for T SNP. However, methylation of the C allele strand markedly enhanced binding and the appearance of an additional nucleo-protein complex. These results link the same cis-regulatory IFNG variant with modulation of DNA strand methylation and transcription factor binding supporting a functional role for rs1861494 gene variant in regulating IFNG expression.

### Example 4

### Materials

Isolation of T cells

Peripheral blood mononuclear cells (PBMC) were isolated from healthy volunteers or IBD patients by separation on Ficoll-Hypaque gradients. CD3′ T cells (PB T) were isolated using CD3-immunomagnetic beads (Miltenyi Biotech, Auburn, Calif.) and were at least 95% pure.

### Study Subjects

Patients with IBD were recruited through the Inflammatory Bowel Disease Center at Cedars-Sinai Medical Center. Diagnoses of Crohn's disease and ulcerative colitis were confirmed using standard clinical, radiological, endoscopic and pathological criteria. All subjects were Caucasian non-Hispanic with the average age of 41 for CD (range 15-78) and 46 for UC (range 11-77) and were genotyped for rs1861494. All genotyping was performed at the Medical Genetics Institute at Cedars-Sinai Medical Center using Infinium technology (Illumina, San Diego, Calif.). Control subjects were healthy individuals, free of medication and with no known personal or family history of autoimmune disease or IBD.

### IFN-γ Assay

IBD T cells were stimulated with anti-CD3 antibody for 24 hours. IFN-γ was measured by an amplified ELISA. Greiner Bio-One (Longwood, Fla.) ELISA plates were coated overnight with 100 μl of 5 μg/ml monoclonal anti-IFN-γ (BD Biosciences, Woburn, Mass.). Samples and standards were added for 24 h followed by addition of 100 μl of 2.5 μg/ml polyclonal biotinylated rabbit anti-IFN-γ (BD Biosciences) for 2 h. This was followed by addition of 100 μl of 1/1000 diluted alkaline phosphatase-conjugated steptavidin (Jackson ImmunoResearch Laboratories, West Grove, Pa.) for 2 h. Substrate, 0.2 mM NADP (Sigma-Aldrich, St. Louis, Mo.) was added for 30 min followed by addition of amplifier (3% 2-propanol, 1 mM iodonitrotetrazolium violet, 75 μg/ml alcohol dehydrogenase, and 50 μg/ml diaphorase; Sigma-Aldrich) for 30 min. Plates were read at 490 nm using an E max plate reader (Molecular Devices, Sunnyvale, Calif.).

### Microbial Antibody Responses

All blood samples were taken at the time of consent and enrolment. Sera were analyzed for expression of ASCA, anti-OmpC, anti-I2 anti-CBir1 antibodies in a blinded fashion by ELISA as previously described (see Targan et al., Antibodies to CBir1 flagellin define a unique response that is associated independently with complicated Crohn's disease, Gastroenterology 2005 128:2020-2028; Mow et al., Association of antibody responses to microbial antigens and complications of small bowel Crohn's disease, Gastroenterology 2004, 126:414-424; and Targan et al., High-titer antineutrophil cytoplasmic antibodies in type-1 autoimmune hepatitis, Gastroenterology 1995, 108:1159-1166, which are incorporated by reference herein in their entirety as though fully set forth). Antibody levels were determined and results expressed as ELISA units (EU/ml) relative to a Cedars-Sinai Laboratory standard that was derived from a pool of patient sera with well-characterized disease found to have reactivity to this antigen.

### Pyrosequencing

DNA was extracted from T cells using a QIAmp DNA isolation kit (Qiagen Inc., Valencia, Calif.). All samples were analyzed in a blinded fashion using the EpigenDx custom pyrosequencing service (EpigenDx, Inc., Hopkinton, Mass.). Briefly, bisulfite treatment of 2 μg of DNA was carried out using the EZ DNA methylation kit (Zymo Research, Orange, Calif.) according to manufacturer's instructions. Hot-start PCR was carried out with HotStart Taq (Qiagen Inc.) using 100 ng of bisulfite treated DNA. PCR and pyrosequencing primers are shown in Table 1.

TABLE 1
PCR and pyrosequencing primers and conditions

| Assay | | | SEQ | | | | |
|---|---|---|---|---|---|---|---|
| CpG | Primers | | ID NO: | PCR Conditions | | | |
| −295, | Fwd | GTGAATGAAGA | 3 | 94° C. | 95° C. 15 s | x 45 | 72° C. |
| −186 | PCR | GTTAATATTTT | | 15 min | 52° C. 30 s | cycles | 5 min |
| | | ATTAGG | | | 72° C. 15 s | | |
| | Rev | Biotin- | 4 | | | | |
| | PCR | TTCCTTTAAAC | | | | | |
| | | TCCTTAAATCC | | | | | |
| | | TT | | | | | |
| −295 | Seq | GTGAATGAAGA | 5 | | | | |
| | | GTTAATATTTT | | | | | |
| | | ATTAGG | | | | | |
| −186 | Seq | GGTGGGTATAA | 6 | | | | |
| | | TGGGTTTG | | | | | |
| | Fwd | GGATTTAAGGA | 7 | 94° C. | 95° C. 15 s | x 45 | 72° C. |
| | PCR | GTTTAAAGGAA | | 15 min | 52° C. 30 s | cycles | 5 min |
| | | A | | | 72° C. 15 s | | |
| −54 | Rev | Biotin- | 8 | | | | |
| | PCR | AAAACAATATA | | | | | |
| | | CTACACCTCCT | | | | | |
| | | CT | | | | | |
| | Seq | TTAAAAAATTT | 9 | | | | |
| | | GTGAA | | | | | |
| Allele- | Fwd | TTTGGGTGATT | 10 | 95° C. | 95° C. 30 s | x 45 | 72° C. |
| specific | PCR | TTGTAAAGTTA | | 15 min | 46° C. 30 s | cycles | 10 min |
| | | GTAAGAGA | | | 72° C. 30 s | | |
| | Rev | CCATTAAAACA | 11 | | | | |
| | PCR | AACAACCTCTC | | | | | |
| | | AT | | | | | |
| | Seq | AAACTACTTCT | 12 | | | | |
| | | CAATACTCC | | | | | |
| T-specific | | ACTACTTCTCA | 13 | | | | |
| | | ATAGTCCCT | | | | | |
| C-specific | | ACTACTTCTCA | 14 | | | | |
| | | ATAGTCCCC | | | | | |

Direct quantification of the ratio of unmethylated to methylated cytosines was determined for each site using Pyro Q-CpG software. The IFNG non-CpG cytosine at site −181 by served as an internal control and revealed that bisulfite conversion of DNA was greater than 95%. Likewise, only slight variability was detected in DNA samples treated with bisulfite on different days. The naïve NK92 cell line demonstrated complete conversion following bisulfite treatment and served as a demethylation control.

### EMSA

PBMC from healthy volunteers were stimulated up to 4 h with PMA/ionomycin and nuclear protein extracts were obtained. Nuclear protein extract (3-6 μg) was incubated at 25° C. with 0.25 mg/ml poly (dI-dC), in 20% glycerol, 5 mM MgCl₂, 2.5 mM EDTA, 2.5 mM DTT, 250 mM NaCl, 50 mM Tris pH 7.5 for 10 min. Oligonucleotides 5′-IRD700-labeled (Integrated DNA Technology, Coraville, Iowa) were then added (250 fmol) and the binding reactions incubated for an additional 30 min. The DNA-protein complexes were separated from unbound probe on a pre-run native 6% polyacrylamide gel in low ionic strength buffer (22.3 mM Tris pH 7.4, 22.3 mM Borate, 0.5 mM EDTA pH 8.0) and analyzed with Odyssey infrared imaging system (Li-Cor Biosciences). The rs1861494 oligonucleotides used were (polymorphic nucleotides are bolded, only upper strand is shown):

(SEQ ID NO: 15)
5′-TCAGTACTCCCTGTGCTTCTTCCTCA-3′,
(SEQ ID NO: 16)
5′-TCAGTACTCCCCGTGCTTCTTCCTCA-3′.

For methylated oligonucleotides, the following sequence was used with either methylation of one or both strands:

(SEQ ID NO: 17)
5′-TCAGTACTCCCC(Me)GTGCTTCTTCCTCA-3′.

### Statistical Analysis

Tests for statistical significance was performed using JMP Statistical Software (SAS Institute GmbH, Heidelberg, Germany) as follows: Test for significance between of rs1861494 SNP and ANCA, IFN-γ secretion and methylation levels was calculated by parametric Student's T test; test of association and trend using Fisher's exact test, cox proportional hazards model and Kaplan-Meier Survival Curves and Log-Rank Test.

### Ethical Considerations

All studies involving human subjects were approved by the Institutional Review Board at Cedars-Sinai Medical Center.

### Example 5

### IFNG Polymorphisms and Methylation Associated With IBD Disease Severity rs1861494 T Allele Carriage Was Increased Among IBD Patients With More Aggressive Disease

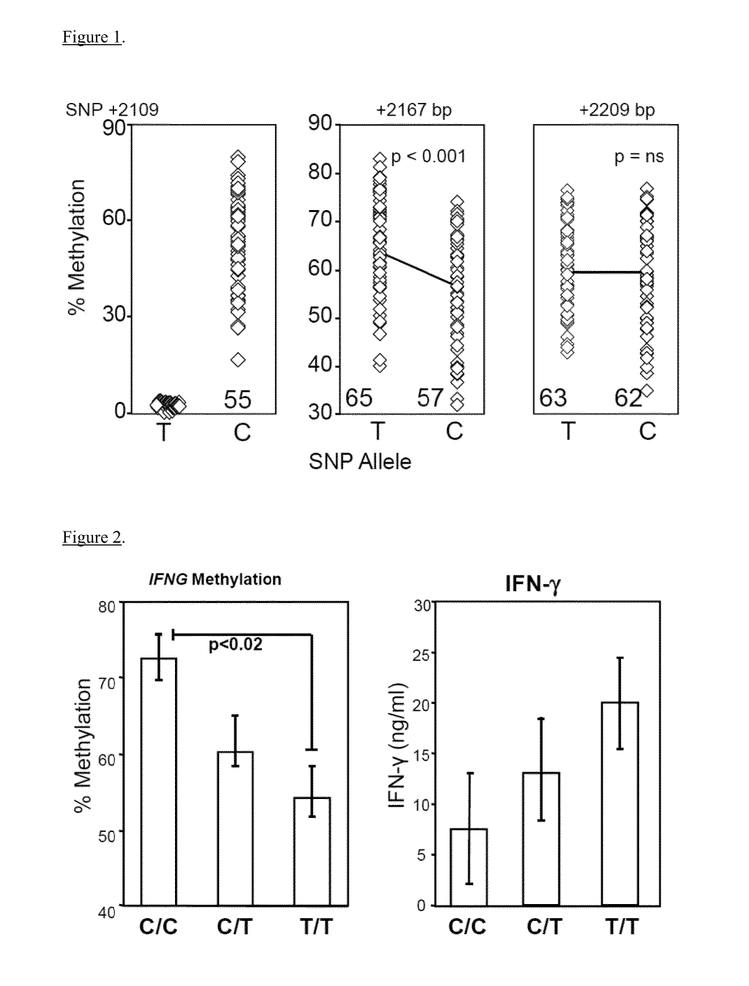

The IFNG +2109 SNP rs1861494 is located within a conserved regulatory region of the third intron of IFNG (FIG. 1). Disease-specific response to microbial and auto-antigens is associated with IBD. Sero-positivity to ANCA is detected in the majority of UC patients, but only a small percentage of CD patients, and is associated with a more aggressive disease phenotype in UC. In order to investigate whether rs1861494 was associated with disease severity in IBD, the IBD patient population was first stratified by disease phenotype and the SNP allele distribution was assessed in individuals with sero-reactivity to ANCA. It was found, in UC populations, the proportion of T (TT and TC) versus C allele (CC) carriers was significantly higher in the ANCA positive compared to ANCA negative patients (p<0.015) (FIG. 2A), and 95% of ANCA positive were T allele carriers (FIG. 2A). It was also found that the rs1861494 T allele carriers were associated with a significant increase in ANCA levels (p<0.001) (FIG. 2B) and an increase in the need for earlier surgical intervention for disease management (p<0.05) (FIG. 2C) in UC patients.

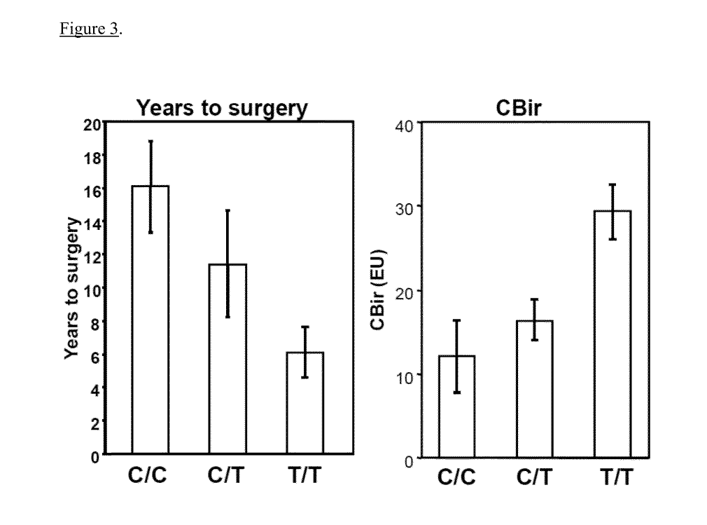

In CD populations, no significant association between rs186194 and ANCA was detected. However, a significant increase of complicated disease behavior, structuring/penetrating phenotype, was found in CD patients carrying the T allele (FIG. 3). No significant association between rs186194 and other CD serological markers (ASCA, OmpC, CBir, and 12) was detected. Thus, although the clinical manifestations differed, T allele carriers were associated with a more severe disease course for both UC and CD.

rs1861494 T Allele Carriage Was Associated With Increased IFN-γProtein Expression.

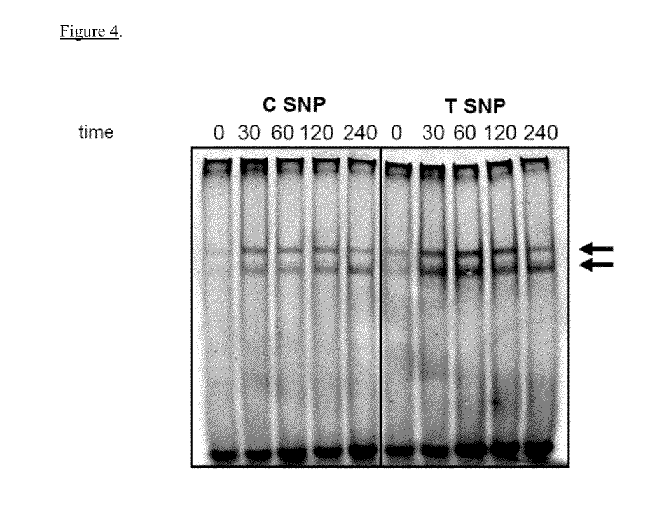

Mucosal expression of IFN-γ is key not only to the development and maintenance of inflammation but, additionally, the absolute levels of IFN-γ influence the severity of disease. Therefore, it was hypothesized that IBD T allele carriers would display a greater level of IFN-γ. Indeed, IFN-γ secretion was enhanced in IBD patients carrying the T allele compared to patients homozygous for the C allele following stimulation of peripheral T cells (FIG. 4).

Methylation of rs1861494 Was Allele-Specific.

The rs1861494 C to T SNP (+2109 bp) introduces a new potential CpG methylation site. An additional two CpG methylation sites reside 52 and 102 by upstream at +2052 and +2007 bp.

DNA methylation is considered an epigenetic marker for expression competency, with reduced methylation usually correlated with enhanced gene expression. It is generally presumed that for most expressed genes each of the two alleles contributes equally. However, introduction of a new potential IFNG CpG methylation SNP might lead to allelic methylation differences, ultimately affecting IFN-γ expression levels. It has been suggested that unequal allele methylation occurs preferentially in sites adjacent to CpG SNPs. In order to ascertain the methylation status of rs1861494, allele/strand specific pyrosequencing methylation analysis was carried out in IBD patients heterozygous for rs1861494. Strand specific methylation levels for each of the two adjacent upstream CpG sites, as well as the rs1861494, were determined. The minor rs1861494 C allele did function as a methylation site (average methylation 60%, FIG. 5A). As expected, no significant methylation was seen for the non CpG common T allele. Furthermore, a significant decrease in the methylation of the +2052 by CpG site was detected when comparing the minor C to the common T allele strands (p<0.001) (FIG. 5B). No difference was seen for the +2007 by CpG site (FIG. 5B). A significant inverse correlation between the de novo IFNG expression and the corresponding overall methylation index (MI) of the promoter IFNG region (−54, −186, and −295 bp) previously demonstrated. Therefore, the relationship between the overall MI of the three sites adjacent to rs1861494 and the MI of the IFNG promoter region was examined. There was a highly significant correlation (p<0.001) between the MI of these two regions for both the T and C alleles (FIG. 5C), suggesting that the methylation status of the rs1861494 region paralleled that of a promoter region known to contribute to regulation of gene expression.

Allele-Specific Nucleo-Protein Binding Was Detected to Methylated rs1861494 DNA-Binding Sites.

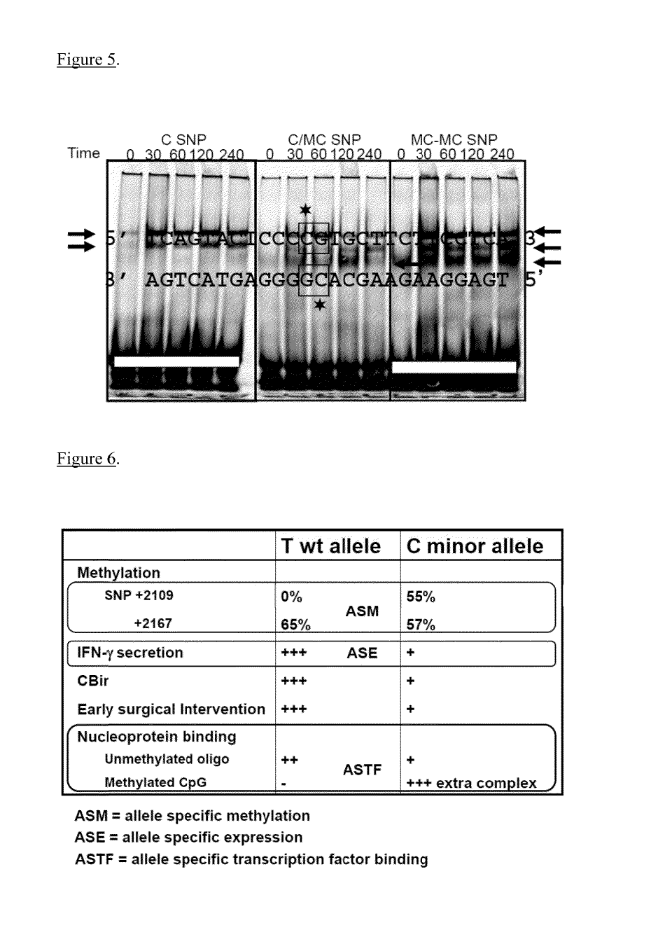

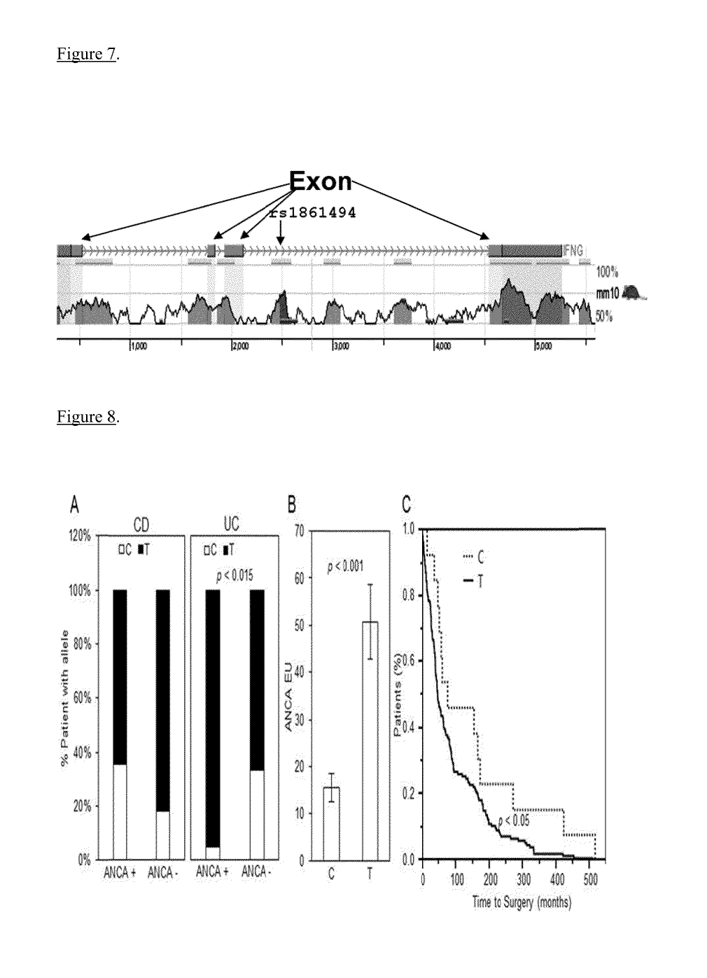

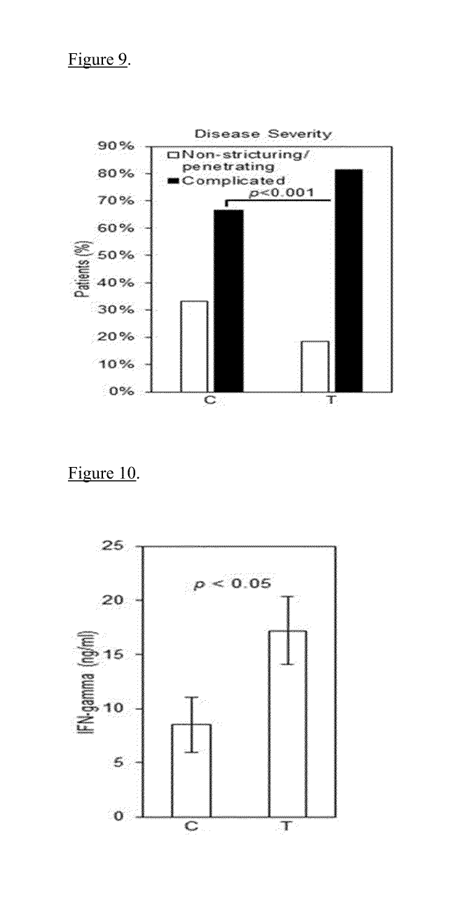

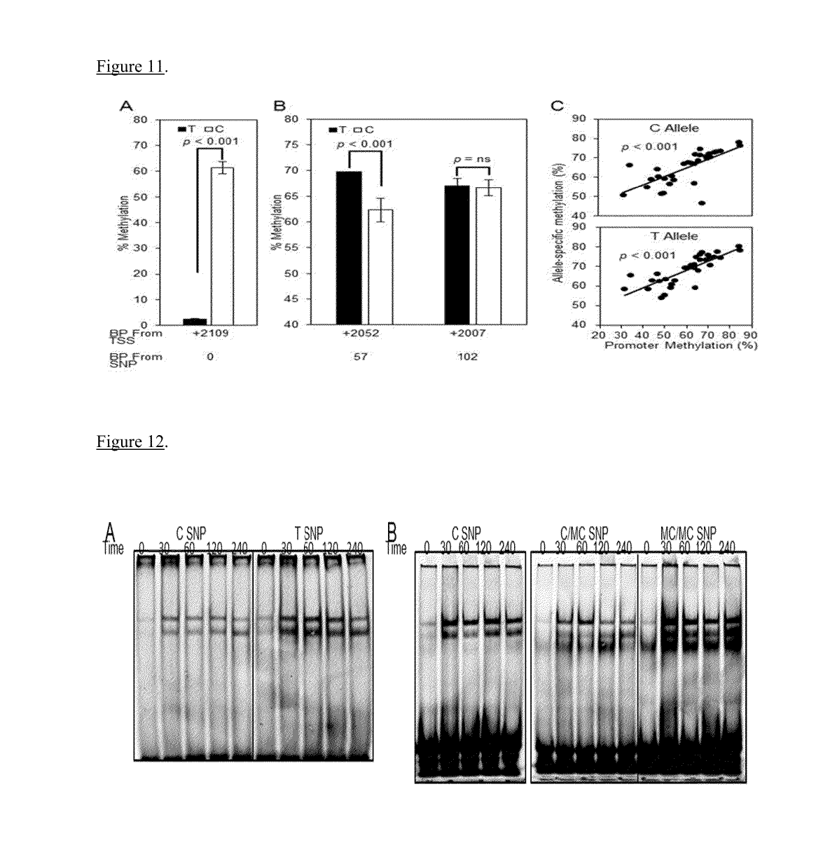

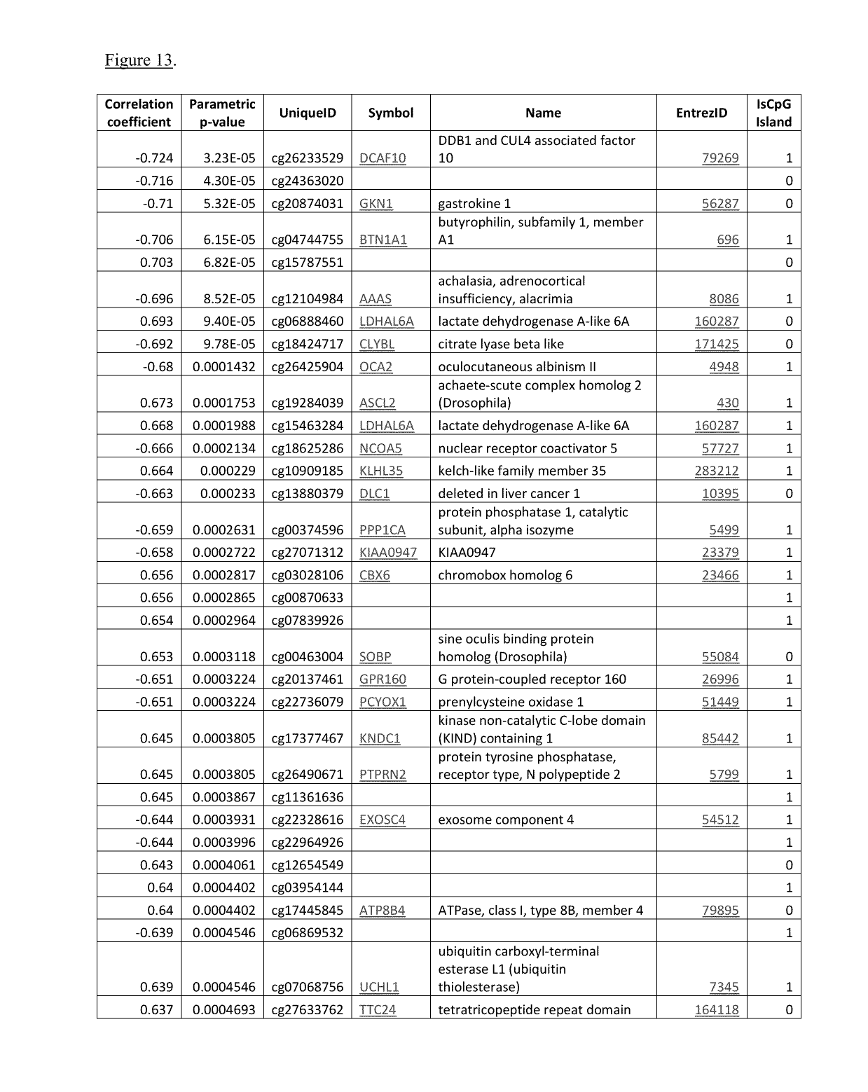

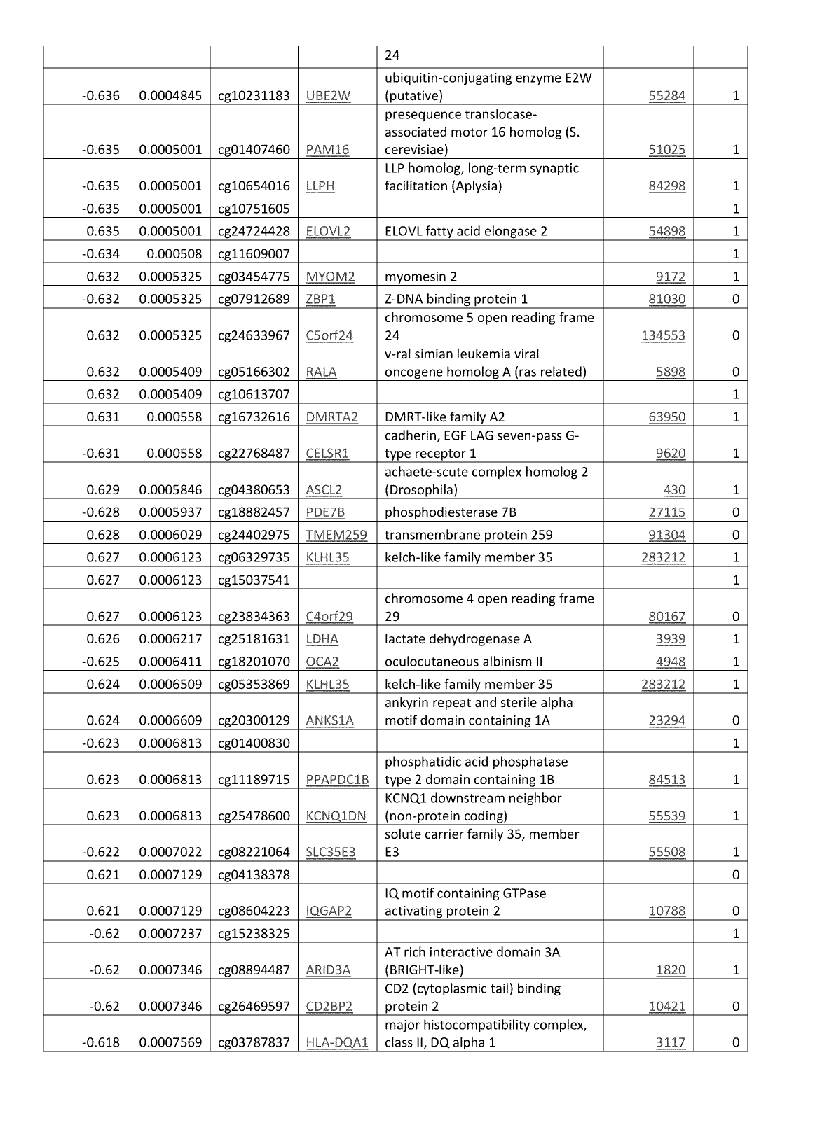

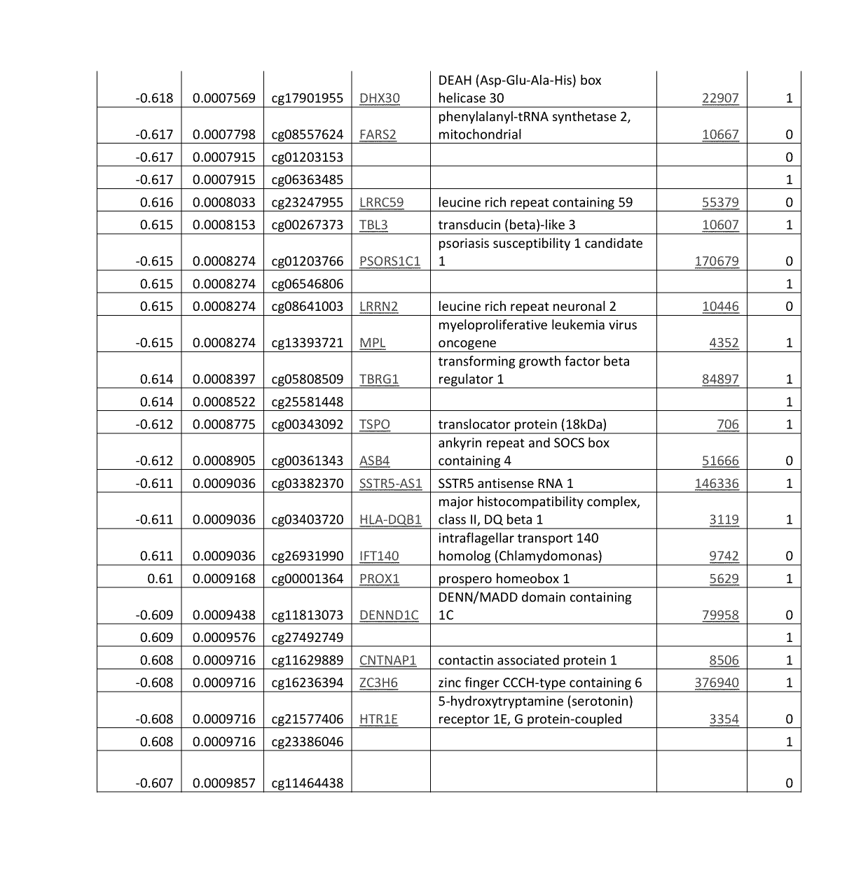

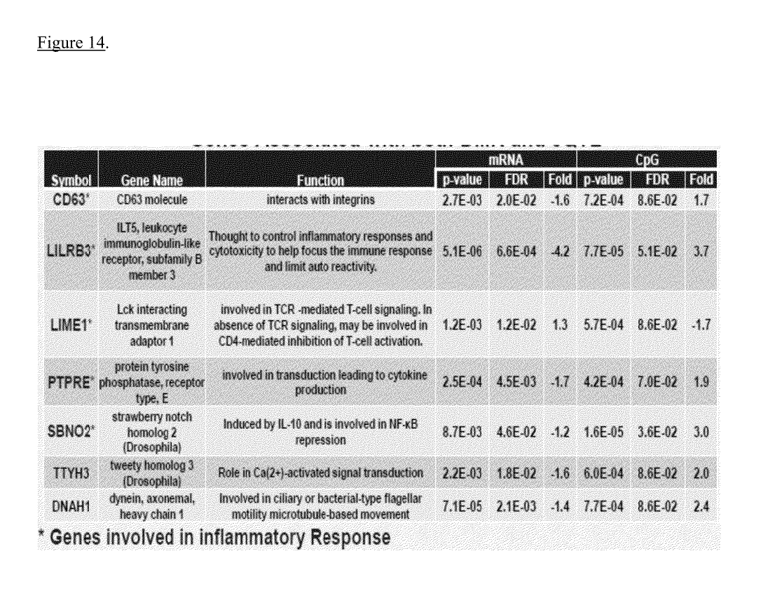

To investigate how the C to T SNP influences transcription factor binding to the SNP rs1861494 region, nuclear extracts were prepared from resting or activated PB T cells. Nucleo-protein binding assays were carried out using dsDNA oligonucleotides corresponding to the common T or minor C SNP allele sequence. Activation-dependent binding of nucleo-protein complex to the T or C SNP oligonucleotide was detected (FIG. 6). Although similar complexes were seen for the common T and minor C oligonucleotides, binding to the T SNP was enhanced compared to the C SNP allele (FIG. 6A).

To investigate how methylation of the rs1861494 CpG influenced transcription factor complex formation, binding assays were set up using a non-methylated, asymmetrically methylated (one strand) C SNP or symmetrically methylated (both strands) C SNP oligonucleotide. Asymmetrical methylation of one C oligonucleotide strand displayed a pattern similar to that seen for the unmethylated oligonucleotide (FIG. 6B, C SNP vs. C/MC SNP). In contrast, symmetric methylation of both C oligonucleotide strands markedly enhanced binding and the appearance of an additional protein complex (FIG. 6B, C/MC SNP vs. MC/MC SNP). These results suggested that both the specific allele sequence and methylation levels could modulate binding of transcription factors to the rs1861494 region and likely regulate IFN-γ expression.

### Example 6

Mucosal expression of IFN-γ and other pro-inflammatory cytokines is critical not only to the development and maintenance of inflammation but, additionally, the absolute amount of IFN-γ appears to modulate the severity of Crohn's disease. This study examined the association between the IFNG rs1861494 T/C polymorphism in IBD. IFNG rs1861494 T allele carriage in IBD patients was associated with enhanced secretion of IFN-γ. In addition, IFNG rs1861494 T allele carriage was associated with clinical/serological parameters indicative a more severe disease course in both UC and CD patients. In UC, T allele carriage was associated with seropositivity and higher levels of the IBD associated autoantibody ANCA. This may be the first study to report a genetic association, outside the HLA region, with ANCA levels. Among patients with medically refractory UC, T allele carriage was associated with a severe disease course that progressed more rapidly toward colectomy. In the CD patient population, T allele carriage was associated with a complicated disease behavior, characterized by stricturing/penetrating phenotype.

These findings are consistent with previously reported studies demonstrating an involvement of IFNG rs1861494 in severity and treatment resistance in other infectious and immune disorders. In tuberculosis, rs1861494 T allele carriage has been associated with susceptibility as well as a more severe microscopy-positive and bacterial positive form of the disease. Furthermore a recent study demonstrated the presence of persistent, elevated levels of IFN-γ in T allele carriers following anti-tuberculosis treatment supporting an association with therapeutic resistance. These data suggests a possible mechanistic role in which elevated IFN-γ expression in T allele carriers may lead to a worse prognosis for the resolution of active disease or accelerated progression to complicated and severe disease. Likewise, in hepatic schistosomiasis, T allele carriage has been associated with poorer control of disease and higher risk of developing severe and extended hepatic fibrosis. In chronic myeloid leukemia, T allele carriage has been associated with poorer response to Imatinib therapy and slower progression of complete cytogenic response.

One of the major challenges in treating IBD is the inter-individual variability of response to therapeutic intervention. The association of rs1861494 T allele carriage with disease severity may help identify patients at risk for aggressive disease before complications occur and thereby reduce the need for surgery. In CD, half of all patients will develop stricturing or penetrating complications within the first 20 years of disease. Most of these patients will go on to require surgery with up to a 61% of CD patient requiring surgery at 10 years. In UC, the expression of ANCA autoantibodies is associated with a complicated disease course and the need for surgical intervention. In fact, a subset of UC patients expressing high levels of ANCA are more likely to develop chronic pouchitis after ileal pouch-anal anastomosis. Moreover, studies have suggested that ANCA reactivity may be associated with poor response to anti-TNF therapy in UC patients. ANCA seronegative UC patients have been shown to be more likely to initially achieve clinical response to anti-TNF therapy than ANCA seropositive patients. Likewise, in children ANCA reactivity has been independently associated with primary nonresponse to anti-TNF therapy. Similar findings have been shown in the CD population in which patients who displayed seropositivity to ANCA were more likely to fail anti-TNF therapy. The initial lack of response to anti-TNF therapy is of clinical importance in CD and even more so in UC. It is likely that lack of primary response to anti-TNF therapy at least in part may reflect that TNF is not the primary mediator of the inflammatory process in this group of patients. Since patients who are seropositive to ANCA are more likely to fail anti-TNF therapy, the present findings of rs1861494 association with disease severity, elevated ANCA and IFN-γ protein levels, suggest that IFN-γ might be what is driving active inflammation in this subset.

IFNG is located on chromosome 12 and is highly conserved through evolution. T cell production of IFN-γ is determined primarily at the transcriptional level through cis and trans factor binding regions. DNA methylation of CpG dinucleotides provides an additional level of regulation and has been associated with transcriptional silencing. The rs1861494 T/C polymorphism is located in the third IFNG intronic region which has been previously reported to possess a T-bet binding site and enhancer activity. Functionally, T allele carriage was correlated with enhanced IFN-γ secretion. The T to C substitution introduces a new CpG dinucleotide sequence that was associated with increased DNA methylation and decreased IFN-γ expression. Electromobility shift assays indicated more intense binding of nuclear protein complex to the T compared to C allele following T cell activation. Moreover, methylation of the CpG sequence within the context of oligonucleotides complementary to the C allele, demonstrated altered nuclear complex formation and the presence of an additional complex. More intense binding of nuclear protein complex to the T and C alleles has been previously reported in nuclear extracts from the Jurkat cell line and additional complex was detected binding the C allele in PHA-blasted T cells. This report is the first to demonstrate methylation dependent binding of nucleo-protein in the context of the rs1861494 C allele and suggests that nucleo-protein binding to this region may be regulated on two levels: one via allele specific affinity for DNA-binding proteins and a second regulated by DNA-methylation-mediated nucleo-protein binding.

Distinct epigenetic IFNG DNA methylation patterns in patients with IBD compared to normal controls has been previously reported. Decreased IFNG methylation is associated with patients requiring surgery and, in UC, is functionally correlated with enhanced IFN-γ secretion and a higher sero-reactivity to what are commonly thought of as “CD-associated” antigens. These data, together with GWAS discovery of UC risk regions on chromosome 12 upstream and downstream of IFNG, suggest that IFNG may play a more central role in the pathogenesis of UC than was originally proposed. One of the fundamental challenges of GWAS is to define the functional consequences of these genetic variations. Surprisingly, many SNPs associated with disease are in fact CpG sites, which are statistically underrepresented in the human genome. Although CpG sites are underrepresented in most non-coding DNA, they are preserved in promoter and other regulatory genomic regions. Thus, in addition to differences detected in DNA methylation patterns in disease, CpG SNPs can introduce sites for allele-specific DNA methylation, the functional consequences of which may affect gene expression via epigenetic mechanisms. Recent studies have suggested that allele specific methylation occurs preferentially in sites adjacent to CpG SNPs. IFNG rs1861494 sits within a region adjacent to two upstream CpG sites. C allele specific methylation at the polymorphic +2109 by site and differential DNA methylation of the adjacent +2052 CpG site were observed, but no allele specific differences were detected for the +2007 by site. More importantly, a significant correlation was observed between the average methylation index across all three sites for both the C and T alleles and the IFNG promoter methylation index. Methylation of the IFNG promoter region has been extensively studied and is believed to play a major role in overall regulation of IFNG expression. While it has been generally reported that enhanced IFN-γ expression corresponds with a decrease in IFNG methylation for some promoter region such as the −22 CNS, it has been demonstrated that the reverse appears to be the case. These finding suggests that epigenetic modulation via methylation of IFNG expression encompasses cooperative interaction across multiple regions.

The data presented here link a genotype-dependent association of the conserved IFNG rs1861494 SNP with allele specific DNA methylation, transcription factor complex formation and IFN-γ protein expression associated with an altered and more aggressive course of disease progression in IBD. Moreover, the data demonstrated an association of rs1861494 with seropositivity and higher levels of ANCA autoantibodies, a biomarker for poorer therapeutic response to anti-TNF therapy. These findings suggest that IFNG rs1861494 may provide new insight into stratifying IBD patients based on whether their mechanism of disease is more dependent of IFN-γ expression and less dependent on TNF. This would be of great prognostic value not only in selecting patients likely to respond to anti-TNF therapy but in potentially identifying patients likely to fail primary anti-TNF therapy and initiate evaluation of other treatment options.

### Example 7

### Methylation and IBD Subgroups

As disclosed herein, the inventors have shown that IBD patients display distinct IFNG methylation, correlated with enhanced IFN-γ secretion and seroreactivity to microbial antigens. As further disclosed herein, the inventors integrated genome wide expression/methylation quantitative trait loci (eQTL/mQTL) and GWAS in order to identify molecular signatures associated with clinical response. They profiled for differentially methylated regions (DMRs) (Illumina450k InfiniumArray) and eQTL(Illuminaexpression array) in CD3+peripheral T cells from 11 CD and 10 UC patients genotyped for the 163 IBD-risk loci. All patients were matched for ethnicity and age and had undergone surgery.

In accordance with various embodiments herein, no genome-wide DMRs were seen between CD and UC patient populations. However, when samples were stratified based on IBD-risk vs. non-risk SNP genotype, 10/163 loci displayed DMRs, of which 2 also showed eQTL: rs6074022 and rs941823. The rs941823 lies within a long non-coding RNA sequence, whereas rs6074022, resides within the CD40 promoter, −7 kb upstream of the transcriptional start site, and was, therefore, chosen for further study. The rs6074022 T (non-risk) to C (risk) SNP introduces a new CpGmethylation site, potentially resulting in altered expression. This was confirmed by real-time PCR using IBD patient mRNA isolated from EBV transformed cell lines. The rs6074022 risk SNP functionally correlated with a significant decrease in CD40 expression (2.3% vs. 1.8% of housekeeping gene EF1α, p<0.05). In addition to altered CD40 gene expression, distinct DMRs (147 CpGsites p<0.001, false discovery rate <0.05) and eQTL(460 transcripts p<0.01, false discovery rate<0.05) were associated with rs6074022. DMRs were enriched in gene bodies or intergenic sites. More than half of DMRs (55%) were defined as regulatory sites. Moreover, DMRs differed between UC and CD patients when stratified for carriage of either the risk and non-risk rs6074022 alleles. CD patients harboring the risk allele were likely to require earlier surgical intervention (p<0.02) for disease management. The rs6074022 functionally impacts not only on CD40 expression but additional target molecules detected by mQTL/eQTLupstream and downstream of the CD40 signaling pathway, i.e. antigen presentation, cytokines and transcriptional regulators. Thus, the inventors have shown a mechanistic role for the rs6074022 SNP in defining altered disease susceptibility and natural history in IBD.

The CD40 IBD risk SNP rs6074022 Displays:
Attenuated CD40 expression
Earlier progression to surgery
rs6074022 SNP Variation is Associated with:
   DMRs between risk vs. non-risk and CD vs. UC
   Differential mRNA expression
CD40 is a key mediator in immune and inflammatory processes. Data suggests a functional association of the CD40 IBD risk rs6074022 with altered epigenetic and expression profiles which may aid in stratifying IBD patients to predict altered pathobiology and course of disease.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of diagnosing susceptibility to an inflammatory bowel disease (IBD) subtype in an individual, comprising:
(a) obtaining a sample from the individual;
(b) assaying the sample to determine the presence or absence of at least one risk genetic variant at the genetic locus of IFNG; and
(c) diagnosing susceptibility to the IBD subtype based on the presence of at least one risk genetic risk variant at the genetic locus of IFNG.

2. The method of claim 1, wherein the assaying the sample comprises using an oligonucleotide probe specific to a risk genetic variant at the genetic locus of IFNG.

3. The method of claim 2, wherein the oligonucleotide probe is labeled with a fluorescent dye.

4. The method of claim 1, wherein the IBD comprises ulcerative colitis.

5. The method of claim 1, wherein the IBD comprises Crohn's disease.

6. The method of claim 1, wherein the IBD is associated with early surgical intervention.

7. The method of claim 1, wherein the IBD is associated with colitis, a small bowel disease phenotype, an aggressive complicating phenotype, an internal penetrating disease phenotype, a stricturing disease phenotype, a fibrostenosing disease phenotype, a fistulating disease phenotype, or a combination thereof.

8. The method of claim 1, wherein the IBD is associated with at least one risk serological marker selected from the group consisting of ANCA, ASCA, anti-Cbir1, anti-I2, and anti-OmpC.

9. The method of claim 1, wherein the at least one risk genetic variant is a “T” allele of SEQ. ID. NO.: 1.

10. The method of claim 9, wherein the at least one risk genetic variant is associated with a lower level of IFNG DNA methylation relative to a healthy subject.

11. The method of claim 6, wherein the at least one risk genetic variant is associated with a higher level of anti-Cbir1 relative to a healthy subject.

12. The method of claim 1, wherein the at least one risk genetic variant is a “C” allele of SEQ. ID. NO.: 1.

13. The method of claim 12, wherein the at least one risk genetic variant is associated with a higher level of IFNG DNA methylation relative to a healthy subject.

14. A method of diagnosing inflammatory bowel disease (IBD) in an individual, comprising:
(a) obtaining a sample from an individual;
(b) assaying the sample to determine the presence or absence of at least one risk genetic variant at the genetic locus of IFNG;
(c) assaying the sample to determine an increase or decrease in IFNG DNA methylation relative to a healthy subject; and
(d) diagnosing IBD in the individual based on the presence of at least one risk genetic variant at the genetic locus of IFNG and an increase in IFNG DNA methylation relative to a healthy subject.

15. The method of claim 14, wherein the IBD comprises Crohn's disease or ulcerative colitis.

16. The method of claim 11, wherein the at least one risk genetic variant is a “T” allele of SEQ. ID. NO.: 1.

17. The method of claim 11, further comprising assaying the sample to identify a high level of anti-Cbir1 relative to a healthy subject.

18. The method of claim 11, wherein the IBD is associated with severe ulcerative colitis conditions.

19. The method of claim 11, wherein the IBD is associated with colitis, a small bowel disease phenotype, an aggressive complicating phenotype, an internal penetrating disease phenotype, a stricturing disease phenotype, a fibrostenosing disease phenotype, a fistulating disease phenotype, or a combination thereof.

20. The method of claim 11, wherein the IBD is associated with at least one risk serological marker selected from the group consisting of ANCA, ASCA, anti-Cbir1, anti-I2, and anti-OmpC.

21. The method of claim 11, wherein the sample comprises a nucleic acid from the individual.

22. The method of claim 11, wherein the sample is a body fluid.

23. The method of claim 19, wherein the body fluid is whole blood, plasma, saliva, mucus, or cheek swab.

24. The method of claim 11, wherein the sample is a cell or tissue.

25. The method of claim 21, wherein the cell is a lymphoblastoid cell line obtained from the individual and transformed with an Epstein Barr virus.

26. The method of claim 21, where in the cell is a mucosal T cell, a lamina propria T cell, or a peripheral blood T cell.

27. A method of treating inflammatory bowel disease (IBD) in an individual, comprising:
(a) obtaining a sample from an individual;
(b) assaying the sample to determine the presence of at least one risk genetic variant at the genetic locus of IFNG;
(c) assaying the sample to determine an aberrant level of IFNG DNA methylation; and
(d) treating the IBD in the individual.

28. The method of claim 27, wherein the IBD comprises Crohn's disease or ulcerative colitis.

29. The method of claim 27, wherein the IBD is associated with early surgical intervention.

30. The method of claim 27, wherein the IBD is associated with colitis, a small bowel disease phenotype, an aggressive complicating phenotype, an internal penetrating disease phenotype, a stricturing disease phenotype, a fibrostenosing disease phenotype, a fistulating disease phenotype, or a combination thereof.

31. The method of claim 27, wherein the at least one risk genetic variant at the genetic locus of IFNG comprises SEQ. ID. NO.: 1.

32. A method of treating an inflammatory bowel disease (IBD) in an individual, comprising:
(a) obtaining a sample from the individual;
(b) assaying the sample to detect the presence or absence of at least one risk genetic variant at the genetic locus of IFNG, and/or assaying the sample to detect an increase or decrease in IFNG DNA methylation relative to a healthy individual; and
(c) treating the IBD in the individual.

33. The method of claim 32, wherein the IBD comprises Crohn's disease (CD) or ulcerative colitis (UC).

34. The method of claim 32, wherein the IBD is associated with colitis, a small bowel disease phenotype, a complicated disease phenotype, an internal penetrating disease phenotype, a penetrating disease phenotype, a stricturing disease phenotype, a fibrostenosing disease phenotype, a fistulating disease phenotype, a severe disease course, or an aggressive disease course, or a combination thereof.

35. The method of claim 32, wherein the individual is a human.

36. The method of claim 32, wherein the sample comprises a nucleic acid from the individual.

37. The method of claim 32, wherein the sample comprises a body fluid, cheek swab, mucus, whole blood, blood, serum, plasma, urine, saliva, semen, lymph, fecal extract, or sputum, or a combination thereof.

38. The method of claim 32, wherein the sample comprises a cell or tissue.

39. The method of claim 38, wherein the cell is a lymphoblastoid cell line obtained from the individual and transformed with an Epstein Barr virus.

40. The method of claim 38, wherein the cell is a mucosal T cell, a lamina propria T cell, or a peripheral blood T cell.

41. The method of claim 32, wherein the at least one risk genetic variant is the “T” allele of SEQ ID NO:1 or SEQ ID NO:2.

42. The method of claim 32, wherein IFNG DNA methylation is IFNG promoter methylation.

43. The method of claim 32, further comprising assaying the sample to detect an increase of INF-γ secretion relative to a healthy individual.

44. The method of claim 32, further comprising assaying the sample to detect an increase or decrease of at least one risk serological marker relative to a healthy individual, wherein the at least one risk serological marker is selected from the group consisting of ANCA, ASCA, anti-Cbir1, anti-I2, and anti-OmpC.

45. The method of claim 32, wherein treating the IBD comprises conducting colectomy on the individual, upon detecting the presence of at least one risk genetic variant at the genetic locus of IFNG and/or a decrease in IFNG DNA methylation relative to a healthy individual.

46. The method of claim 32, wherein treating the IBD comprises administering a TNF signaling inhibitor to the individual, upon detecting the absence of at least one risk genetic variant at the genetic locus of IFNG and/or an increase in IFNG DNA methylation relative to a healthy individual.

47. The method of claim 46, wherein the TNF signaling inhibitor comprises an anti-TNF antibody.

48. The method of claim 46, wherein the TNF signaling inhibitor comprises infliximab, adalimumab, certolizumab, certolizumab pegol, golimumab, etanercept, or onercept, or a combination thereof.

49. The method of claim 46, wherein the TNF signaling inhibitor is administered topically, intravascularly, intravenously, intraarterially, intramuscularly, subcutaneously, intraperitoneally, intranasally, or orally.

50. The method of claim 46, wherein the TNF signaling inhibitor is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof.

51. The method of claim 46, wherein the TNF signaling inhibitor is administered about 1-3 times per day, 1-7 times per week, or 1-9 times per month.

52. The method of claim 46, wherein the TNF signaling inhibitor is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

53. A method of treating an inflammatory bowel disease (IBD) in an individual, comprising:
(a) genotyping the individual for a risk genetic variant at the genetic locus of IFNG; and
(b) if the individual is positive for the risk genetic variant, conducting colectomy on the individual, and if the individual is negative for the risk genetic variant, administering a TNF signaling inhibitor to the individual.

54. A method of treating an inflammatory bowel disease (IBD) in an individual, comprising:
(a) obtaining a sample from the individual;
(b) contacting the sample with an oligonucleotide probe specific to a risk genetic variant at the genetic locus of IFNG;
(c) forming an allele-specific hybridization complex between the oligonucleotide probe and the risk genetic variant;
(d) detecting the allele-specific hybridization complex; and
(e) if the allele-specific hybridization complex is detected, conducting colectomy on the individual, and if the allele-specific hybridization complex is not detected, administering a TNF signaling inhibitor to the individual.

55. The method of claim 53 or 54, wherein the individual is a human.

56. The method of claim 53 or 54, wherein the risk genetic variant is the “T” allele of SEQ ID NO:1 or SEQ ID NO:2.

57. The method of claim 54, wherein the oligonucleotide probe is labeled with a fluorescent dye, and wherein detecting the allele-specific hybridization complex comprises detecting fluorescence signal from the oligonucleotide probe.

58. The method of claim 54, wherein the oligonucleotide probe comprises a reporter dye and a quencher dye.

59. The method of claim 57, further comprising conducting PCR amplification after forming the allele-specific hybridization complex.

60. The method of claim 57, wherein detecting the allele-specific hybridization complex comprises detecting the electrophoretic mobility of the allele-specific hybridization complex.
